(19) United States
(12) Patent Application Publication
(10) Pub. No.: US 2015/0096555 A1
(43) Pub. Date: Apr. 9, 2015

(54) ADMINISTRATION METHOD FOR A MEDICAL SPRAY INHALER AND THE MEDICAL SPRAY INHALER

(71) Applicants: Ditmanson Medical Foundation Chia-Yi Christian Hospital, Chia-Yi City (TW); Kun Shan University, Tainan City (TW)

(72) Inventors: Song-Hao WANG, Vienna, VA (US); Pei-Hua CHANG, Tainan (TW); Cheng-Ren CHEN, Chia-Yi City (TW); Ming-Shian LIN, Chia-Yi City (TW)

(73) Assignees: Ditmanson Medical Foundation Chia-Yi Christian Hospital, Chia-Yi City (TW); Kun Shan University, Tainan City (TW)

(21) Appl. No.: 14/045,439

(22) Filed: Oct. 3, 2013

(51) Int. Cl. A61M 15/00

(52) U.S. Cl. CPC A61M 15/009; A61M 15/0091; A61M 15/0008
USPC 128/200.17; 128/200.23

(57) ABSTRACT

An administration method for a medical spray inhaler and the medical spray inhaler, which utilizes an inhaling sensing unit to sense an air flow change for generating an inhaling signal after a user inhales by an inhaler body. A control unit is then used to receive the inhaling signal and calculate a delay time. After the delay time, the control unit outputs a control instruction to control a medicine container disposed on the inhaler body to spray a medical spray and concurrently records the ventilation volume per inhalation of the user.

### FIELD OF THE INVENTION

The present invention relates to an administration method for a medical spray inhaler and the medical spray inhaler.

### BACKGROUND OF THE INVENTION

Taiwan patent number 1310905 entitled “DOSE INHALER, RELATED RECORDING DEVICE AND METHOD FOR DOSE INHALER” discloses a dose inhaler for recording an effective medication time and frequency of a patient. Taiwan patent number 1224513 entitled “DOSE COUNTER FOR MEDICAMENT INHALER” discloses an accurate and consistent medication dose metering system that dispenses dry powdered medicament in discrete amounts or doses for patient inhalation, a pressure relief system that manages pressure within a medicament reservoir of the inhaler to ensure consistently dispensed doses, and a dose counting system indicating the number of doses dispensed from the inhaler.

While the user uses the conventional inhaler, the time of inhaling and pressing the medicine container are usually inconsistent, such that most of the medical spray stay in the oral cavity and cannot be inhaled into the deep of the throat directly and causes a bad treatment efficacy. Furthermore, doctors usually examine the healed breath condition of the patients by patients' dictations or by other precision instruments so as to determine whether the use frequency of the medical spray inhaler should be increased or decreased, which consumes more medical costs.

### SUMMARY OF THE INVENTION

The present invention provides an administration method for a medical spray inhaler, which comprises steps of: (a) using an inhaling sensing unit to sense an air flow change and generate an inhaling signal when a user inhales by the inhaler body; (b) using a control unit to receive the inhaling signal, and then using a timing module to calculate a delay time after the control unit receives the inhaling signal; and (c) using the control unit to output a control instruction to control a medicine container disposed on the inhaler body to spray a medical spray after the delay time.

Preferably, the control unit controls a pressing unit to press the medicine container to spray the medical spray.

Preferably, the inhaling sensing unit senses an inhaling air speed and an inhaling time of an inhalation of the user. The control unit calculates a ventilation volume according to the inhaling air speed, the inhaling time, and a know measure of an air intake and saves the ventilation volume in a recording module.

The present invention further provides a medical spray inhaler allowing a medicine container to be set therein for use and using the administration method for a medical spray inhaler, which comprises an inhaler body including an inhaling part being tunneled through and a medicine fixing seat connected to the inhaling part. The medicine container is disposed on the medicine fixing seat. The medical spray inhaler further comprises an inhaling sensing unit disposed on the inhaling part and a control unit electrically connected to the inhaling sensing unit. The control unit includes a timing module. Wherein, an inhaling signal is generated by using the inhaling sensing unit to sense an air flow change in the inhaling part of the inhaler body, the control unit receives the inhaling signal, then the timing module calculates a delay time, and the control unit outputs a control instruction to control the medicine container to spray a medical spray after the delay time.

Preferably, the medical spray inhaler comprises a pressing unit disposed on the medicine fixing seat and electrically connected to the control unit. Wherein, the pressing unit is disposed corresponding to the medicine container, thereby the control unit controls the pressing unit in order to press the medicine container to spray the medical spray.

Preferably, the inhaling sensing unit includes a room communicated with an inner space of the inhaling part, a fan having a plurality of fan blades and disposed in the room, a magnet disposed on at least one of the fan blades, an air intake formed on a wall of the room for being communicated with the room and an external environment, and a magnetic sensing chip disposed on an interior wall of the room. The control unit is electrically connected to the magnetic sensing chip.

Preferably, the pressing unit includes a casing sleevedly disposed on the medicine fixing seat of the inhaler body, a slide track disposed in the casing, a gear rack penetrated into the slide track and aiming at the medicine container, and a gear wheel engaged with the gear rack. The gear wheel is sleeved on a rotating shaft of a motor. The motor is electrically connected to the control unit.

Preferably, the control unit includes a recording module. The inhaling sensing unit senses an inhaling air speed and an inhaling time in the inhaling part of the inhaler body. The control unit then calculates a ventilation volume according to the inhaling air speed, the inhaling time, and a known measure of the air intake and saves the ventilation volume in the recording module.

The present invention also provides a medical spray inhaler using the administration method for a medical spray inhaler, which comprises an inhaler body including an inhaling part being tunneled through and a medicine fixing seat connected to the inhaling part, an inhaling sensing unit disposed on the inhaling part, and a control unit electrically connected to the inhaling sensing unit. The control unit includes a timing module. Wherein, an inhaling signal is generated by using the inhaling sensing unit to sense an air flow change in the inhaling part of the inhaler body, the control unit receives the inhaling signal, then the timing module calculates a delay time, and the control unit outputs a control instruction after the delay time.

Preferably, the medical spray inhaler further comprises a pressing unit disposed on the medicine fixing seat and electrically connected to the control unit. Wherein, the pressing unit is disposed corresponding to a medicine container for allowing the control unit to control an operation of the pressing unit.

Preferably, the inhaling sensing unit includes a room communicated with an inner space of the inhaling part, a fan having a plurality of fan blades and disposed in the room, a magnet disposed on at least one of the fan blades, an air intake formed on a wall of the room for being communicated with the room and an external environment, and a magnetic sensing chip disposed on an interior wall of the room. The control unit is electrically connected to the magnetic sensing chip.

Preferably, the pressing unit includes a casing sleevedly disposed on the medicine fixing seat of the inhaler body, a slide track disposed in the casing, a gear rack penetrated into the slide track, and a gear wheel engaged with the gear rack. Wherein, the gear wheel is sleeved on a rotating shaft of a motor, and the motor is electrically connected to the control unit.

Preferably, the control unit includes a recording module. The inhaling sensing unit senses an inhaling air speed and an inhaling time in the inhaling part of the inhaler body, and the control unit calculates a ventilation volume according to the inhaling air speed, the inhaling time, and a known measure of the air intake and saves the ventilation volume in the recording module.

The efficiencies of the present invention:

1. The inhaling sensing unit generates the inhaling signal, and the control unit outputs a control instruction to control the medicine container to spray the medical spray after a delay time, so that the medicine is allowed to reach the deep of the throat for performing the best treatment efficiency.

2. The recording module records the ventilation volume per inhalation of the user, so the medical staffs are able to determine the treatment efficacy according to the recorded ventilation volume and adjust the use frequency for the user.

### BRIEF DESCRIPTION OF THE DRAWINGS

FIG. 1 is a perspective view showing an appearance of a medical spray inhaler of a preferred embodiment in accordance with the present invention;

FIG. 2 is a section view of the FIG. 1;

FIG. 3 is a flow chart showing a method of the preferred embodiment in accordance with the present invention;

FIG. 4 is a schematic view showing a pressing unit of the preferred embodiment in accordance with the present invention that is pressing a medicine container to spray a medical spray; and

FIG. 5 is a partially enlarged view of the FIG. 4 showing an operation of an inhaling sensing unit of the preferred embodiment in accordance with the present invention.

### DETAILED DESCRIPTION OF THE PREFERRED EMBODIMENTS

The present invention will become clearer in light of the following detailed description of an illustrative embodiment of this invention described in connection with the drawings. It is intended that the embodiments and drawings disclosed herein are to be considered illustrative rather than restrictive. In addition, it should note that the like elements are denoted by the similar reference numerals throughout disclosure.

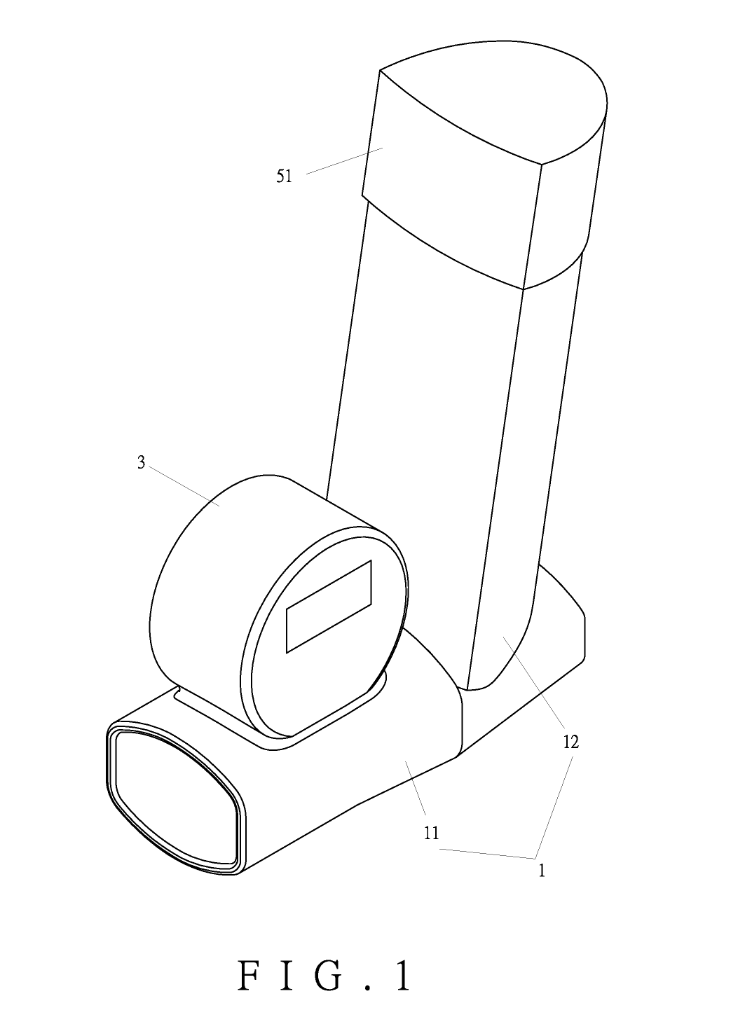

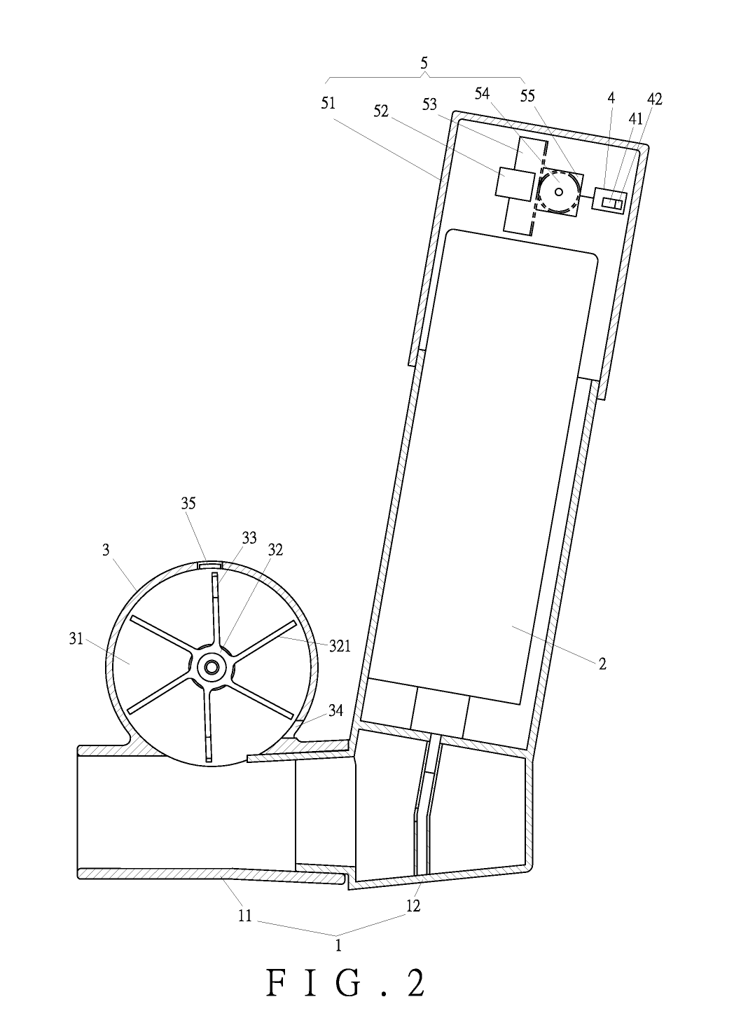

Referring to FIGS. 1 and 2, a structure of a medical spray inhaler of the present invention comprises: an inhaler body 1 including an inhaling part 11 being tunneled through and a medicine fixing seat 12 connected to the inhaling part 11, wherein a medicine container 2 is disposed on the medicine fixing seat 12; an inhaling sensing unit 3 disposed on the inhaling part 11, wherein the inhaling sensing unit 3 includes a room 31 communicated with an inner space of the inhalant part 11, a fan 32 having a plurality of fan blades 321 and disposed in the room 31, a magnet 33 disposed on at least one of the fan blades 321, an air intake 34 formed on a wall of the room 31 for being communicated with the room 31 and an external environment, and a magnetic sensing chip 35 disposed on an interior wall of the room 31; a control unit 4 electrically connected to the magnetic sensing chip 35 of the inhaling sensing unit 3 (it is easy to understand for the profession, and the connection is omitted in the figure), wherein the control unit 4 includes a timing module 41 and a recording module 42; and a pressing unit 5 including a casing 51 sleevedly disposed on the medicine fixing seat 12 of the inhaler body 1, a slide track 52 disposed in the casing 51, a gear rack 53 penetrated into the slid track 52 and disposed corresponding to the medicine container 2, and a gear wheel 54 engaged with the gear rack 53, wherein the gear wheel 54 is sleeved on a rotating shaft of a motor 55, and the motor 55 is electrically connected to the control unit 4.

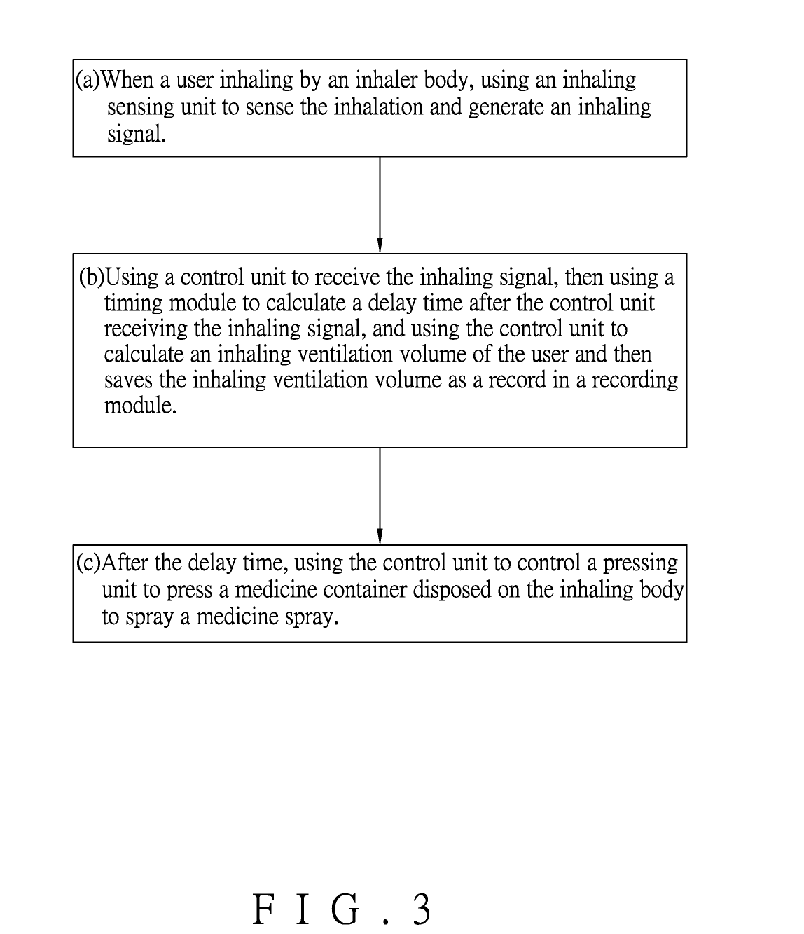

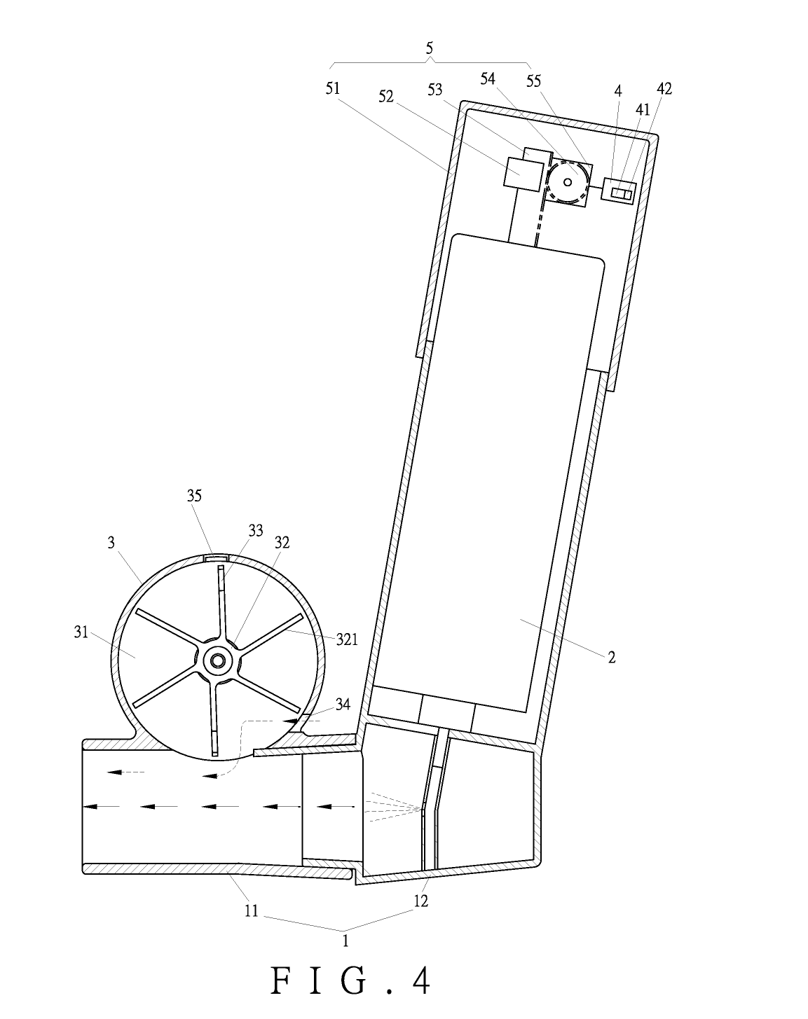

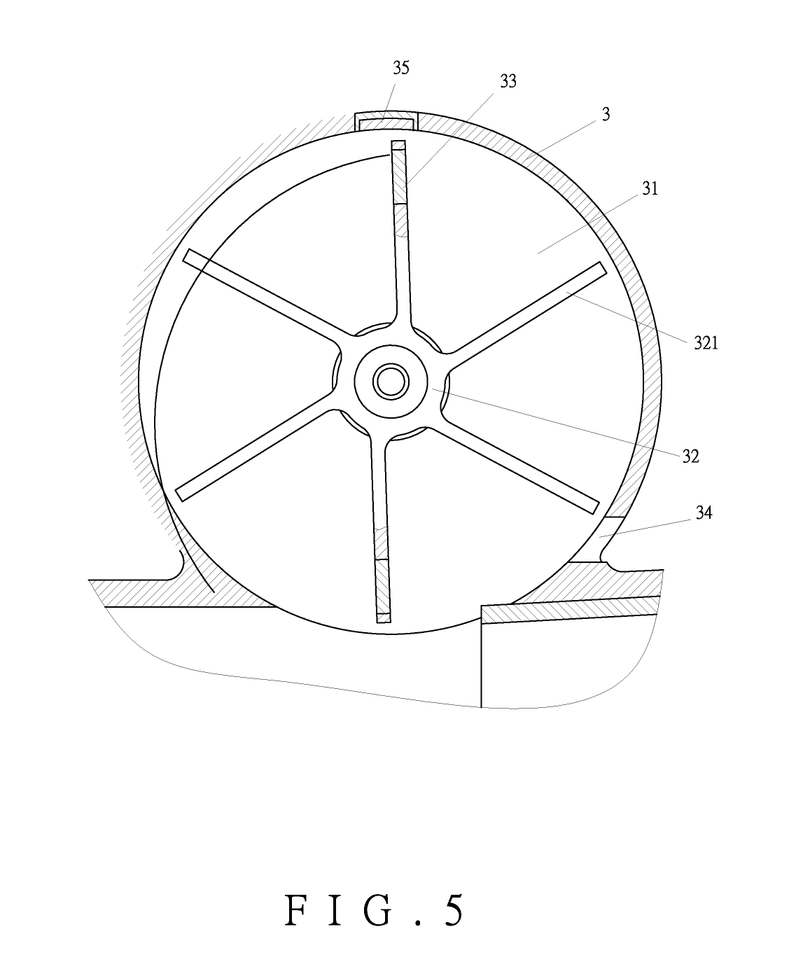

When in use, referring to FIGS. 3, 4, and 5, an administration method for the medical spray inhaler comprises the steps of:

(a) A user inhales by the inhaling part 11 of the inhaler body 1. The room 31 of the inhaling sensing unit 3 is communicated with an inner space of the inhaling part 11, so the air is inhaled from the air intake 34 and actuates at least one of the fan blades 321 of the fan 32 to rotate. The magnetic sensing chip 35 then generates an inhaling signal by sensing a magnetic change, which is caused by the rotation of the fan blade(s) 321, of the magnet 33 on the fan blade(s) 321. Meanwhile, a rotation speed of the fan 32 is derived from the cyclic magnetic change of the magnet 33, and the rotation speed of the fan 32 is able to be converted into an air speed of the air inhaled from the air intake 34. Furthermore, according to the measure of the air intake 34 and the inhaling time, the control unit 4 is able to calculate a flow rate of the air inhaled from the air intake 34, namely the ventilation volume of which the user inhales each time, and the ventilation volume will be recorded in the recording module 42.

(b) The control unit 4 receives the inhaling signal. After the control unit 4 receives the inhaling signal, the timing module 41 calculates a delay time which is set variously according to different users, for example, a difference between the delay time set for old people and children is about 0.3 to 0.5 seconds.

(c) After the delay time, the control unit 4 controls the motor 55 of the pressing unit 5 to operate. The motor 55 drives a rotation of the gear wheel 54, and the gear wheel 54 drives the gear rack 53 to move in the slide track 52 to press the medicine container 2 and allow the medicine container 2 to spray the medical spray. However, the present invention is not restricted to the above-mentioned pressing mechanism. A pressing mechanism utilizing a piston moving in a cylinder or other pressing mechanisms also can be selected as a mechanism for pressing the medicine container 2. According to the above-mentioned administration method, the medical spray is sprayed at the time that the user inhales, so that the medical spray is able to be fully inhaled by the user without remaining too much medicine in the oral cavity, thereby performing a best treatment efficiency of the medicine.

Furthermore, the ventilation volumes of which the user inhales every time are saved in the recording module 42, so that the doctor can estimate the treatment efficacy, adjust the medicine use frequency, or consider other treatment methods such as changing the medication according to the record saved in the recording module 42.

While we have shown and described the preferred embodiment in accordance with the present invention, it should be clear to those skilled in the art that further embodiments may be made without departing from the scope of the present invention.

What is claimed is:

1. An administration method for a medical spray inhaler comprising steps of:
(a) using an inhaling sensing unit to sense an air flow change and generate an inhaling signal when a user inhales by said inhaler body;
(b) using a control unit to receive said inhaling signal, and then using a timing module to calculate a delay time after said control unit receives said inhaling signal; and
(c) using said control unit to output a control instruction to control a medicine container disposed on said inhaler body to spray a medical spray after said delay time.

2. The administration method for a medical spray inhaler as claimed in claim 1, wherein in said step (c), said control unit controls a pressing unit to press said medicine container to spray said medical spray.

3. The administration method for a medical spray inhaler as claimed in claim 1, wherein said inhaling sensing unit senses an inhaling air speed and an inhaling time of an inhalation of said user, and said control unit calculates a ventilation volume according to said inhaling air speed, said inhaling time, and a known measure of an air intake and saves said ventilation volume in a recording module.

4. A medical spray inhaler allowing a medicine container to be set therein for use and using the administration method for a medical spray inhaler of claim 1, wherein said medical spray inhaler comprises an inhaler body including an inhaling part being tunneled through and a medicine fixing seat connected to said inhaling part, and said medicine container is disposed on said medicine fixing seat; characterized in that said medical spray inhaler further comprises:
an inhaling sensing unit disposed on said inhaling part; and
a control unit electrically connected to said inhaling sensing unit, said control unit including a timing module, wherein an inhaling signal is generated by using said inhaling sensing unit to sense an air flow change in said inhaling part of said inhaler body, said control unit receives said inhaling signal, then said timing module calculates a delay time, and said control unit outputs a control instruction to control said medicine container to spray a medical spray after said delay time.

5. The medical spray inhaler as claimed in claim 4 further comprises a pressing unit disposed on said medicine fixing seat and electrically connected to said control unit, wherein said pressing unit is disposed corresponding to said medicine container, thereby said control unit controls said pressing unit in order to press said medicine container to spray said medical spray.

6. The medical spray inhaler as claimed in claim 4, wherein said inhaling sensing unit includes a room communicated with an inner space of said inhaling part, a fan having a plurality of fan blades and disposed in said room, a magnet disposed on at least one of said fan blades, an air intake formed on a wall of said room for being communicated with said room and an external environment, and a magnetic sensing chip disposed on an interior wall of said room; said control unit being electrically connected to said magnetic sensing chip.

7. The medical spray inhaler as claimed in claim 4, wherein said pressing unit includes a casing sleevedly disposed on said medicine fixing seat of said inhaler body, a slide track disposed in said casing, a gear rack penetrated into said slide track and aiming at said medicine container, and a gear wheel engaged with said gear rack, wherein said gear wheel is sleeved on a rotating shaft of a motor; said motor being electrically connected to said control unit.

8. The medical spray inhaler as claimed in claim 4, wherein said control unit includes a recording module, said inhaling sensing unit senses an inhaling air speed and an inhaling time in said inhaling part of said inhaler body, and said control unit calculates a ventilation volume according to said inhaling air speed, said inhaling time, and a known measure of said air intake and saves said ventilation volume in said recording module.

9. A medical spray inhaler using the administration method for a medical spray inhaler of claim 1, which comprises:
an inhaler body including an inhaling part being tunneled through and a medicine fixing seat connected to said inhaling part;
an inhaling sensing unit disposed on said inhaling part; and
a control unit electrically connected to said inhaling sensing unit, said control unit including a timing module, wherein an inhaling signal is generated by using said inhaling sensing unit to sense an air flow change in said inhaling part of said inhaler body, said control unit receives said inhaling signal, then said timing module calculates a delay time, and said control unit outputs a control instruction after said delay time.

10. The medical spray inhaler as claimed in claim 9 further comprises a pressing unit disposed on said medicine fixing seat and electrically connected to said control unit, wherein said pressing unit is disposed corresponding to a medicine container for allowing said control unit to control an operation of said pressing unit.

11. The medical spray inhaler as claimed in claim 9, wherein said inhaling sensing unit includes a room communicated with an inner space of said inhaling part, a fan having a plurality of fan blades and disposed in said room, a magnet disposed on at least one of said fan blades, an air intake formed on a wall of said room for being communicated with said room and an external environment, and a magnetic sensing chip disposed on an interior wall of said room; said control unit being electrically connected to said magnetic sensing chip.

12. The medical spray inhaler as claimed in claim 9, wherein said pressing unit includes a casing sleevedly disposed on said medicine fixing seat of said inhaler body, a slide track disposed in said casing, a gear rack penetrated into said slide track, and a gear wheel engaged with said gear rack; wherein, said gear wheel is sleeved on a rotating shaft of a motor, and said motor is electrically connected to said control unit.

13. The medical spray inhaler as claimed in claim 9, wherein said control unit includes a recording module, said inhaling sensing unit senses an inhaling air speed and an inhaling time in said inhaling part of said inhaler body, and said control unit calculates a ventilation volume according to said inhaling air speed, said inhaling time, and a known measure of said air intake and saves said ventilation volume in said recording module.
